(19) United States
(12) Patent Application Publication
(10) Pub. No.: US 2007/0122487 A1
(43) Pub. Date: May 31, 2007

(54) (Poly(acryloyl-hydroxyethyl starch)-plga composition microspheres

(75) Inventors: Patrick Deluca, Lexington, KY (US); Ge Jiang, Durham, NC (US); Byung Woo, Schaumburg, IL (US)

Correspondence Address: BUCHANAN, INGERSOLL & ROONEY PC, POST OFFICE BOX 1404, ALEXANDRIA, VA, 22313-1404, US

(73) Assignee: UNIVERSITY OF KENTUCKY RESEARCH FOUNDATION, Lexington, KY (US)

(21) Appl. No.: 10/549,760

(22) PCT Filed: Mar. 19, 2004
(86) PCT No.: PCT/US04/08399

§ 371 Date: Dec. 21, 2006

Related U.S. Application Data
(60) Provisional application No. 60/455,590, filed on Mar. 19, 2003.

(51) Int. Cl. A61K 38/22; A61K 38/28; A61K 38/18; A61K 38/21; A61K 9/14; A61K 9/50

(52) U.S. Cl. 424/490; 514/2; 514/3; 514/15; 424/85.4; 424/46

(57) ABSTRACT

The present invention relates to a composite microsphere system comprising poly(D,L-lactide-co-glycolide) (PLGA), poly(acryloyl hydroxyethyl starch) (AcHES), and a pharmaceutically effective amount of a biologically active compound. The active compound may be, for example, an insulin, an interferon, a luteinizing hormone-releasing hormone (LHRH) analog, a somatostatin and/or derivatives thereof, a calicitonin, a parathyroid hormone (PTH), a bone morphogenic protein (BMP), an erythropoietin (EPO), an epidermal growth factor (EGF) or a growth hormone. This invention also relates to methods of using the composite microspheres, and methods of preparing same.

### FIELD OF THE INVENTION

The present invention relates to a composite microsphere system comprising poly(D,L-lactide-co-glycolide) (PLGA), poly(acryloyl hydroxyethyl starch) (AcHES), and a pharmaceutically effective amount of a biologically active compound. The active compound may be, for example, an insulin, an interferon, luteinizing hormone-releasing hormone (LHRH) analogs, somatostatin and derivatives thereof, calicitonin, parathyroid hormone (PTH), bone morphogenic protein (BMP), erythropoietin (EPO), epidermal growth factor (EGF) or growth hormone. This invention also relates to methods of using the composite microspheres, and methods of preparing same.

### BACKGROUND OF THE INVENTION

Biodegradable microspheres are effective as delivery systems for biologically active peptides and proteins. Sustained release characteristics of microspheres reduce the need for frequent administrations and enhance patient compliance by maintaining in vivo drug levels in the therapeutic range. Additional advantages of this drug delivery system include biocompatibility, controlled biodegradability, absorbability, non-toxicity degradation products, potential for sustained release and targeting and ease of administration.

Nevertheless, there are problems associated with the use of currently known microsphere systems for protein delivery. For example, protein instability has been observed during the preparation of protein-loaded microspheres (See Li et at., J. Control Release, 68:41-51 (2000)). Usually, an aqueous protein solution is dispersed in an organic polymer solution by using a homogenizer or sonicator to create a water-in-oil emulsion. However, the exposure of protein to organic solvent often has adverse effects on the stability of the proteins. During drug release, the absorption of protein on the hydrophobic polymer matrices and a low pH generated during the polymer degradation process could cause degradation of the entrapped protein.

A second problem is presented by the initial burst release of protein drugs from microspheres. The fast diffusion of protein drugs located on the surface of internal pores and channels formed by the evaporation of solvent and water during the microsphere preparation contributes to the burst release. One approach to overcome this problem is the chemical modification or physical blending of PLGA with hydrophilic monomers and polymers such as polyethylene glycol (PEG), poly(ethylene-co-vinyl acetate), and polyvinyl alcohol (PVA) (See Nam et al., J. Microencapsul., 16:625-637 (1999)). Another approach is physical encapsulation of protein-loaded hydrophilic particles or hydrogels into a PLGA matrix.

However, in the preparation of microspheres, protein drugs are often exposed to large amounts of organic solvent and multiple freezing-thawing or heating-cooling processes during protein loading in the primary hydrophilic particles.

Despite success with small peptides, PLGA microspheres are known to have a number of problems with delivery of proteins and polypeptides. Structural or conformational changes of proteins during microsphere manufacturing, storage and release are known (See Cleland et al., Pharm. Res., 14: 420-5 (1997); and Crotts et al., J. Microencapsul., 15: 699-713 (1998)). For example, during microsphere preparation, an aqueous protein solution is often dispersed in an organic polymer solution by using a homogenizer or sonicator to create a water-in-oil emulsion. The exposure of proteins to organic solvent and high sheer has adverse effects on the integrity of the proteins (See Nihani et al., J. Colloid Interface Sci., 173:55-65 (1995)). During drug storage and release protein unfolding and aggregation often occurs due to interaction of protein molecules with the hydrophobic polymeric surface. Moreover, the low pH generated during polymer erosion causes chemical degradation of entrapped proteins.

PLGA microspheres are also considered to have an inconsistent release profile. For example, a high initial burst effect within 24 hours followed by a plateau and then culminating in incomplete release is often seen. This initial burst is undesirable, because high initial drug release is not suitable for therapeutic proteins due to the risk of side effects from high serum levels. Attempts have been made to improve protein stability and release kinetics of the PLGA system by changing the physico-chemical properties of the polymer. For example, both chemical derivation and physical blending of PLGA with hydrophilic monomers and polymers such as polyethylene glycol, poly(ethylene-co-vinyl acetate) and polyvinyl alcohol have been reported (See Péan et al., Pharm. Res., 16:1294-1299 (1999)). However, to date there is little understanding of the in vitro and in vivo behavior of composite microspheres and essentially no in vivo pharmacological evaluation of therapeutic proteins from these composite systems.

### SUMMARY OF THE INVENTION

An object of the present invention is to provide a composite microsphere system comprising poly(D,L-lactide-co-glycolide) (PLGA), poly(acryloyl hydroxyethyl starch) (AcHES), and a pharmaceutically effective amount of a biologically active compound, wherein the biologically active compound is a polypeptide having a molecular weight of about 200 to about 160,000 Daltons. Preferably, the biologically active compound is an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone.

Another object of the present invention provides a drug formulation comprising a composite microsphere system comprising poly(D,L-lactide-co-glycolide) (PLGA), poly(acryloyl hydroxyethyl starch) (AcHES), and a pharmaceutically effective amount of a biologically active compound, wherein the biologically active compound is an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone and a pharmaceutically acceptable vehicle.

A further object of the present invention provides a method for the sustained release delivery of a therapeutic compound to a subject comprising: administering to the subject a composite microsphere system comprising poly(D,L-lactide-co-glycolide) (PLGA), poly(acryloyl hydroxyethyl starch) (AcHES), and a pharmaceutically effective amount of a biologically active compound, wherein the biologically active compound is an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone.

Preferably, the subject is suffering from a condition Which may be treated and/or cured by the administration of an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone. Preferably, the subject is a vertebrate or an invertebrate organism. More preferably, the subject is a canine, a feline, an ovine, a primate, an equine, a porcine, a caprine, a camelid, an avian, a bovine, an amphibian, a fish, or a murine organism. Most preferably, the primate is a human.

Preferably, the microspheres are in a pharmaceutically acceptable vehicle and the microspheres are administered topically. Topical administration may include inhalation or nasal administration. Also preferably, the microspheres may be administered parenterally. Preferably, the microspheres may be administered intramuscularly.

A further object of the present invention provides a method of preparing a composite microsphere system comprises incorporating a biologically active ingredient selected from the group consisting of an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone into AcHES hydrogel microparticles, encapsulating the resulting AcHES hydrogel microparticles containing the biologically active ingredient into a PLGA matrix. Preferably, the AcHES hydrogel microparticles containing the biologically active ingredient are incorporated into the PLGA matrix using either solvent extraction, solvent evaporation, spray drying, freeze drying or a combination thereof.

### BRIEF DESCRIPTION OF THE DRAWINGS

FIG. 1 shows SEM micrographs of insulin-loaded AcHES-PLGA composite microspheres (a), interior structure of a fractured microsphere (b), (the arrows indicate embedded AcHES microparticles) and freeze dried AcHBS hydrogel microparticles (c).

FIG. 2 shows a HPLC chromatogram of an insulin sample isolated from composite microspheres by ACN extraction and the intact insulin standard.

FIG. 3 shows a characterization of insulin integrity in the composite microspheres. (a) shows a SDS-PAGE with DTT. Lane 1 shows the molecular weight marker, Lane 2 shows the bovine insulin standard; Lanes 3 and 4 shows an insulin samples. (b) shows a MALDI-TOF MS of insulin extracted from composite in comparison to intact standard.

FIG. 4 shows in vitro release of insulin from composite microspheres in glycine buffer at 37° C. Sonication levels are indicated by ( ).

FIG. 5 shows serum glucose suppression in diabetic rats treated with insulin loaded composite microsphere batches (a) and (b).

FIG. 6 shows serum insulin level of (a) and (b) treated diabetic rats.

FIG. 7 shows blood glucose suppression of multiple dosing treatment of insulin loaded composite microspheres (n=8, Dose 80 IU/rat).

FIG. 8 shows serum insulin level of multiple dosing treatment of insulin loaded composite microspheres (n=8, Dose 80 IU/rat).

### DETAILED DESCRIPTION OF THE PREFERRED EMBODIMENTS OF THE INVENTION

A. Definitions

In general, the terms in the present application are used consistently with the manner in which those terms are understood in the art.

By “micro” is meant a particle having a diameter of from nanometers to micrometers.

By “composite microsphere” is meant a microsphere matrix formed of at least two different polymeric materials. In the present invention, the polymeric materials include PLGA particles and AcHES particles. A “composite” is an aggregation of microspheres made as described herein, bound by materials known to those skilled in the art for this purpose.

By “pharmaceutically acceptable vehicle” is meant the vehicles in which the composite microspheres are suspended or distributed. The vehicles may include excipients and auxiliaries which facilitate processing of the active compounds into preparations which can be used for delivery to the site of action, such as oil adjuvants.

By “effective amount” or “dose effective amount” or “therapeutically effective amount” is meant an amount of an agent which modulates a biological activity of the proteins of the invention.

II. Introduction

The present invention provides a composite microsphere system comprising poly(D,L-lactide-co-glycolide) (PLGA), poly(acryloyl hydroxyethyl starch) (AcHES) and a pharmaceutically effective amount of a biologically active compound. Preferably, the biologically active compound is a polypeptide having a molecular weight of about 200 to about 160,000 Daltons. More, preferably, the active compound is an insulin, an interferon, a luteinizing hormone-releasing hormone (LHRH) analogs, a somatostatin and/or somatostatin derivative, a calicitonin, a parathyroid hormone (PTH), a bone morphogenic protein (BMP), an erythropoietin (EPO), an epidermal growth factor (EGF) or a growth hormone. This novel composite microsphere system based on PLGA and poly(acryloyl hydroxyethyl starch) (AcHES) was developed using bovine serum albumin and horseradish peroxidase (HRP). These composite microspheres show a more favorable and complete release than conventional PLGA microspheres. The present invention also provides methods of using and preparing the composite microsphere system.

Composite Microsphere System and Formulations

The composite microsphere system of the present invention comprises poly(D,L-lactide-co-glycolide) (PLGA), poly(acryloyl hydroxyethyl starch) (AcHES), and a pharmaceutically effective amount of a biologically active compound. The biologically active compound may include, but is not limited to, an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), epidermal growth factor (EGF) or a growth hormone. Types of insulin appropriate for use with the present invention include, but are not limited to, human insulin and bovine insulin. Types of interferons appropriate for use with the present invention include, but are not limited to, alpha-interferon, beta-interferon and gamma-interferon. Types of calcitonin appropriate for use with the present invention include eel, human and salmon.

The present invention also provides a drug formulation comprising a composite microsphere system comprising poly(D,L-lactide-co-glycolide) (PLGA), poly(acryloyl hydroxyethyl starch) (AcHES), a pharmaceutically effective amount of a biologically active compound, and a pharmaceutically acceptable vehicle. Preferably, the biologically active compound is an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone.

The pharmaceutically acceptable vehicles may include, but are not limited to, any excipients and auxiliaries which facilitate processing of the active compounds into preparations which can be used pharmaceutically for delivery to the site of action. For example, suitable formulations for parenteral administration may include aqueous solutions of the active compounds in water-soluble form, for example, water-soluble salts. Optionally, the suspension may also contain stabilizers.

The composite microsphere system and formulation are appropriate for parenteral or topical administration. Specifically, topical routes may include nasal administration or inhalation. If indicated, the different types of compositions and formulations may be used simultaneously to achieve systemic administration of the active ingredient.

The composite microsphere system and formulations of this invention may be used alone or in combination with each other, or in combination with other therapeutic or diagnostic agents, especially in cases where an insulin, an interferon, a luteinizing hormone-releasing hormone (LHRH) analog, a somatostatin and/or somatostatin derivative, a calicitonin, a parathyroid hormone (PHI), a bone morphogenic protein (BMP), an erythropoietin (EPO), an epidermal growth factor (EGF) or a growth hormone is indicated for treatment. In certain preferred embodiments, the compounds of this invention may be co-administered along with other compounds typically prescribed for these conditions according to generally accepted medical practice. The agents of the present invention can be provided alone, or in combination with other agents that modulate a particular pathological process. As used herein, two or more agents are said to be administered in combination when the two agents are administered simultaneously, or are administered independently in a fashion such that the agents will act contemporaneously.

Methods of Treatment Using the Composite Microsphere System

The present invention also provides a method for the sustained release delivery of a therapeutic compound to a subject comprising administering to the subject a composite microsphere system comprising AcHES, a polymeric material such as PLGA, and a pharmaceutically effective amount of a biologically active compound contained within the AcHES microsphere. Preferably, the biologically active compound is an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone. The subject may be a vertebrate or an invertebrate organism. Specifically, the subject may be a canine, a feline, an ovine, a primate, an equine, a porcine, a caprine, a camelid, an avian, a bovine, an amphibian, a fish, or a murine organism. Preferably, the primate is a human.

The amount of active biologically active compound incorporated in the microsphere system delivery device varies widely depending on the particular agent, the desired effect and the time span over which it takes the matrix to release the compound. The upper and lower limits on the amount of the compound to be incorporated into the device can be determined empirically by comparing microspheres containing a range of compound. The dosage administered to a patient will be dependent also on the age, health, and weight of the recipient, type of concurrent treatment (if any), frequency of treatment, and the nature and stage of disease and the nature of the effect desired. The microsphere system of the methods of the present invention are preferably administered topically, via inhalation or nasal administration, or by parenteral administration.

Microspheres produced as described above are small enough to be injected through a standard gauge needle under the skin or into the peritoneum for subsequent release of incorporated drug. Adhesion of the microspheres to the peritoneum aids in localizing release of the incorporated drug. Microspheres can also be implanted or injected intramuscularly and subcutaneously for immunization or other purposes where slower release into the bloodstream is desirable.

The microsphere system of the present invention may be administered in order to target a specific organ. For example, the lung may be targeted by administering the microsphere system via inhalation. The lung may also be targeted by using intravenous (IV) administration, so long as the microsphere size is appropriate. Specifically, the microspheres should be about 8-30 microns in size in order to lodge in the capillaries of the lungs. Microspheres as large as 130 microns may be administered via a hypodermic needle for intramuscular and subcutaneous administration.

Microspheres may be suspended in a suitable pharmaceutical vehicle for administration using methods appropriate for the vehicle and site of administration. Vehicles may include phosphate buffer, saline, gelatin, a cellulosic agent, a surfactant or an oil adjuvant. Suitable pharmaceutical vehicles are known to those skilled in the art and commercially available.

The diseases, disorders and conditions which may be treated by the methods and systems of the present invention include diabetes. The present invention may be used to deliver insulin to the body in the treatment of diabetes. Patients suffering from diabetes often have to take one or more types of insulin for the rest of their lives. The method of treatment of the present invention may be convenient and safe in delivering insulin to diabetic patients.

The present invention may also be used to deliver human growth hormone to a subject in need of same. The present invention may further be used to deliver interferon to the body in the treatment of diseases including, but not limited to, cancer, hepatitis and conditions related to Acquired Immune Deficiency Syndrome (AIDS). In addition, the present invention may be used to deliver LHRH analogs, somatostatin and derivatives thereof, calcitonin, PTH, BMP, EPO and EGF to a subject in need thereof.

The biologically active ingredient may also include, but is not limited to, melanocyte-stimulating hormone (MSH), thyrotropin-releasing hormone (TRH), luteinizing hormone (LH), follicle stimulating hormone (FSH), vasopressin, oxytonin, parathyroid hormone, glucagon, gastrin, secretin, pancreozymin, cholecystokinin, angiotensin, lactogen, human chononic gonadotropin (HCG), enkephalin, endorphin, kyotorphin, tuftsin, thymopoietin, thymosin, thymosthymlin, thymic humoral factor (TBF), serum thymic factor (FTS), tumor necrosis factors (TNF), colony stimulating factors (CSF), motilin, dynorphin, bombesin, neurotensin, caerulein, bradykinin, urokinase, asparaginase, kallikrein, substance P, nerve growth factor, blood coagulation factor VIII and IX, lysozyme chloride, polymyxin B. colistin, gramicidin, bacitracin, protein synthesin-stimulating peptide, gastric inhibitory polypeptide (GIP), vasoactive intestinal polypeptide (VIP), platelet-derived growth factor (PDGF), growth hormone-releasing factor (GRF), and bone morphagenetic protein (BMP).

Methods of Preparing the Composite Microsphere System

The present invention provides a method for the preparation of a composite microsphere system of, comprising incorporating an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone into AcHES hydrogel microparticles and encapsulating the resulting AcHES hydrogel microparticles containing an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone into a PLGA matrix. The AcHES hydrogel microparticles containing the active ingredient are preferably incorporated into the PLGA matrix using either solvent extraction, solvent evaporation, spray drying, freeze drying or a combination thereof.

A more homogenous distribution of hydrogel particles throughout the PLGA matrix will retard the initial release of the active ingredient and make the subsequent release dependant on polymer hydration and mass loss. As an alternate to sonication, surfactants, such as Tween 20, may be used to reduce the burst of the active ingredient.

### EXAMPLE 1

### Preparation and Characterization of Microspheres

Hydrophilic starch-based hydrogel particles containing model proteins were prepared by a simple swelling procedure. The protein-loaded hydrogel particles were then encapsulated in PLGA microspheres to form a hydrogel-PLGA combined composite microspheres, using a solvent extraction or evaporation method. Bovine serum albumin (BSA) and horseradish peroxidase (HRP) were used as model protein drugs. Physicochemical characteristics and in vitro protein release of microspheres were studied to establish poly(acrylol hydroxyethyl starch)-PLGA (AcHES-PLGA) composite microspheres as a protein delivery system.

Poly(D,L-lactide-co-glycolide) (PLGA). copolymer ratio of 50:50 (lactic/glycolic; MW 28,000) and Resomer® RG503H were supplied by Boehringer Ingelheim (Ingelheim, Germany). Hydroxyethyl starch was obtained from Dupont Pharmaceuticals (Wilmington, Del.). Acryloyl chloride was purchased from Aldrich Chemicals Company, Inc. (Milwaukee, Wis.). BSA and polyvinyl alcohol (PVA, MW 30,000-70,000) were obtained from Sigma Chemical Co. (St. Louis, Mo.), HRP, 1-Step™ Slow TMB-ELISA and micro-BCA protein assay kit were obtained from Pierce (Rockford, Ill.).

Preparation of AcHES Hydrogel Particles

An acrylic acid ester of hydroxyethyl starch (AcHES) was prepared. 20 g of HES was dissolved in 60 mL dimethyl acetamide and an appropriate amount (2-10 mL) of distilled acryloyl chloride, based on desired degree of derivatization (DD, number of vinyl groups introduced on the hydroxyethyl group in every unit of HES polymer chain), and an equimolar amount of triethylamine were added slowly to the HES solution. The reaction mixture was precipitated by adding 200 mL of precooled acetone, and the precipitate was dissolved in 50 mL deionized water. The solution was transferred to a dialysis tubing with a molecular weight cutoff of 14,000 and dialyzed against deionized water with frequent change of water for 48 hours. The dialyzed solution was freeze-dried. The DD was determined by proton-NMR spectroscopy (22).

AcHES polymer (7:3 mixture of DD=0.14 and 0.25) was dissolved in 0.1M phosphate-buffered saline (PBS) (pH 7.4) to make a 30% (w/v) solution, and ammonium peroxidisulfate was added to the solution to form a dispersed phase. The dispersed phase was added to 50 mL mineral oil containing 0.3% of Sorbitan Sesquioleate while stirring to form a w/o emulsion. N,N,N′,N′,tetramethylethylenediamine (300 μL) was added to the emulsion to initiate the polymerization reaction followed by continuous stirring at room temperature for 1 hour. The suspension containing polymerized droplets was poured into precooled hexane while sonicating. The AcHES hydrogel particles were collected by centrifugation at 1000 rpm for 5 minutes, washed twice with hexane and ethanol, rinsed with deionized water several times and freeze-dried.

Preparation of PLGA-AcHES Composite Microspheres

The PLGA-AcHES composite and conventional PLGA microspheres were prepared by a modified solvent extraction of evaporation method with 5-10% target loading of BSA and 5% for HRP. 25-50 mg of BSA and 25 mg HRP were dissolved in 0.23 mL of 0.1 M PBS (pH 7.4). The protein solutions were added to AcHES particles (10% or total polymer weight), and the particles were allowed to swell for 5 min with vortex mixing at room temperature. Twenty percent (w/w) PLGA (90% of total polymer weight) in methylene chloride was added to the swollen AcHES particles and vortexed for 3 minutes at room temperature to form a (protein in hydrogel)/(polymer in solvent) dispersion. This primary dispersion was then added to precooled 100 mL 6% PVA solution and stirred by a Silverson mixer (Silverson, Chesham Bucks, England) at 5000 rpm for 1 minute. The resulting secondary suspension was transferred to 1 L deionized water and stirred gently for 3 hours at room temperature to remove the organic solvent and solidify the polymer. The microspheres were washed with water and freeze-dried. For the conventional PLGA microspheres, a primary emulsion was prepared by mixing the protein solutions with 20% PLGA solution, and then the emulsion was added to 6% PVA solution while stirring at 5000 rpm. The resulting suspension was transferred to 1 L deionized water and stirred gently for 3 hours at room temperature to remove the organic solvent and fabricate the polymer. The microspheres were washed with water and freeze-dried.

Particle Characterization and Morphology of Microspheres

PLGA-AcHES microspheres (10 mg) were dispersed in 10 mL 0.1% Tween 80 solution. The particles were sized by laser diffractometry by using a Malvern 2600 laser size. The average particle size was expressed as the volume mean diameter in micrometers.

The surface morphology and internal structure of fractured microspheres were examined by scanning electron microscopy (SEM) (model S800; Hitachi, Tokyo, Japan) after palladium-gold coating of the microsphere samples on a aluminum stub.

Drug Loading Efficiency

Ten-milligram protein-loaded PLGA-AcHES microspheres were hydrolyzed in a mixture of 0.9 mL of 1 M NaOH and 0.1 mL PBS with vigorous shaking at room temperature for 1 hour. Protein standard solutions (0.1 mL) were also hydrolyzed after adding 0.9 mL 1 M NaOH using the same procedures. After hydrolysis, 1 mL 0.9 M HCl was added to neutralize the sample solutions. Protein concentrations were determined by a micro-BCA total protein assay method. The loading efficiency was calculated by the actual protein loading to the theoretical loading of protein in PLGA-AcHES microspheres, based on the amount used in the microsphere preparation.

In Vitro Protein Release

Microspheres were weighed and placed in 15-mL centrifuge tubes containing PBS with 0.02% sodium azide as a preservative. The tubes were incubated at 37° C. with occasional shaking. At designated times, samples were collected, and the release medium was replaced with fresh PBS. The samples were assayed by a micro-BCA method or by using a fluorescence spectrophotometer (model F2000: Hitachi) at excitation and emission wavelengths of 280 and 350 nm. The two assay methods showed comparable results for in vitro release samples.

Protein Stability

The structural integrity of proteins extracted from the microspheres was characterized by size-exclusion chromatography (SEC) and sodium dodecyl sulfate poly(acrylamide) gel electrophoresis (SDS PAGE). Ten-mg of microspheres were dissolved in 0.1 mL CH₂Cl₂. Proteins were extracted from the polymer solution by addition of 1 ml. 0.1 M PBS followed by agitation for 1 hour.

SEC was performed by using a Biosep SEC-S2000 column (4.6×300 mm; Phenomenex, Torrance, Calif.). The mobile phase was 0.1 M phosphate buffer (pH 7.0) containing 0.02% sodium azide. The flow rate was 0.5 mL/min, and the detection wavelength was UV 280 nm. The injection volume was 20 μL.

SDS-PAGE was carried out in the presence of 0.1% SDS using a 9% slab gel prepared by a gel casting and electrophoresis unit (Mini-Protean® H electrophoresis system; Bio-Rad. Hercules, Calif.). Protein samples and standards were treated with SDS-PAGE sample buffer containing SDS and dithiothreitol for 3 minutes at 95° C., and electrophoresis was performed at a constant voltage of 200 V. Protein bands on the gel were stained with Coomassie Brilliant Blue.

The enzymatic activity of HRP was determined by using a substrate solution, 1-Step™” Slow TMB-ELISA. 5 μL of HRP standard solutions (1-10 μg/mL), and samples were mixed with 0.4 mL of the substrate solution and incubated at room temperature for 2 minutes. The absorbency at 450 nm was measured, and the specific activity of samples was calculated by using an activity calibration curve obtained from standard HRP solutions.

Characterization of Microspheres

The AcHES particles for protein entrapment possess a submicrometer particle size of average 0.14 μm, a low bulk density of 0.05 g/cc, and high specific surface area. In addition, the AcHES hydrogel particles showed fast and good swelling properties. The hydrated particles showed about an 1]-fold larger particle diameter, and were 10.5-fold heavier, than the dry particles. This suggests that the hydrophilic starch-based hydrogel could absorb a large amount of aqueous drug solution inside and protect the drugs from degradation due to solvent and polymer interactions during the microsphere preparation and drug release.

The average particle size of the composite microsphere ranged from 39.1 to 93.1 μm. A similar mean particle size was observed from different protein contents of BSA-loaded microspheres that showed the particle size ranged from 39 to 52 μm. HRP-loaded microspheres were larger than BSA-loaded microspheres prepared by the same target drug load. The particle size of PLGA-AcHES composite microspheres was affected by two preparation parameters. The first was the concentration of PLGA in the solvent and the mixing speed of the primary suspension in the continuous phase. The second was the size of particle increased with increase in polymer concentration and decrease in stirring rate.

Drug Encapsulation Efficiency

BSA was encapsulated successfully in the composite and PLGA microspheres with 88-101% drug-loading efficiency. The composite microspheres prepared with 5% target BSA load showed a higher encapsulation efficiency than 10% target load. For the PLGA microspheres, a similar drug encapsulation efficiency was obtained from the different target BSA load. However, there was no significant difference in drug encapsulation efficiency with different target drug loads. HRP-loaded microspheres showed lower loading efficiencies of 40.5-50.9% compared to that of the BSA-loaded microspheres. The drug encapsulation efficiency increased with increasing PLGA polymer concentration in the disperse phase and PVA in the continuous phase. Higher viscosity achieved by increasing polymer and PVA concentrations could minimize diffusion of protein from the disperse phase to continuous phase during the fabrication of microspheres, and may also have resulted in more condensed PLGA matrices around entrapped aqueous protein droplets. The drug encapsulation efficiency was not improved by increasing the ratio of hydrogel to PLGA.

In Vitro Release

BSA- and HRP-loaded composite microspheres showed about 40% initial protein release in 24 hours followed by slow release for 21 days. An accelerated and nearly linear release was observed between 21 and 42 days. This three-stage release pattern could be explained as follows. The initial release is due to diffusion of proteins from AcHES hydrogel particles located near the microsphere surface, and from interchannels and inner pores formed by solvent removal during the microsphere solidification process. A second stationary phase observed between the initial release and the third phase, in which release was increased by erosion of polymer matrices. The conventional PLGA microspheres showed about 20% more initial release followed by a very slow release for 42 days. This suggests that PLGA-AcHES composite structure could suppress the initial burst results of encapsulated protein compared to PLGA microspheres. The cross-linked starch-based polymer structure of AcHES could retard the diffusion of protein drugs from the inside of hydrogel particles through the PLGA matrix. In addition, the swelled hydrogel particles could prevent the penetration of release media into the channels and pores in the PLGA matrices.

At the third phase of erosion-controlled release, PLGA microspheres showed a very slow release for 42 days, compared to gradual release of the composite microspheres. As the PLGA polymer degraded by hydrolysis in the aqueous release media, the properties of microspheres changed, the molecular weight of polymer decreased, acid number of degraded polymer increased, and hydration increased. The spherical microsphere particles stuck together and turned into a sticky gel-like cake, and finally, the hydrated polymer became completely soluble. The swelling, hydration, and gelling of PLGA matrices may block the diffusion channels in the microspheres and, consequently, decrease the release of relatively large protein molecules located in the core of the microspheres.

In addition, an increase of acid number of the polymer causes more protein binding to the polymer, and the protein-polymer interaction may be another reason for slow release from PLGA microspheres. Compared to PLGA microspheres for the composite microspheres, dissolution of the PLGA domains could expose the entrapped drug-containing AcHES hydrogel particles to the release media, and the exposed hydrogel could release more drug molecules with little or no interaction with the PLGA polymer. As a result, the composite microspheres showed more favorable in vitro release than the conventional PLGA microspheres for protein drug delivery.

Protein Stability and Activity

The proteins extracted from the PLGA-AcHES composite microspheres showed good stability without structural integrity changes. The protein extracted showed same retention time of native HRP without the trace of protein aggregates degradation products. These results suggest that the preparation process of the composite microspheres did not affect the structural integrity of proteins. The enzymatic activity of HRP was also examined to determine the activity loss of the protein during the microsphere preparation process and in vitro drug release. HRP was more stable in the composite microspheres than PLGA microspheres. HRP extracted from the composite and PLGA microspheres showed 80.9 and 61.5% specific activity, respectively. The different activity between HRP extracted and released suggests that the loss of activity mainly occurred during the extraction of protein from the microspheres. However, in addition to stabilizing the protein during the preparation process, the composite microspheres protected entrapped HRP during in vitro release. HRP from the composite microspheres showed much higher enzymatic activity (P<0.05) than HRP from PLGA microspheres after 7 days of incubation an the release medium. The results suggest that the starch-based hydrogel particles in the composite microspheres could stabilize protein drugs from the degradation, aggregation, and loss of activity not only during the microspheres preparation process but also during the release.

Thus, a biodegradable microsphere system has been developed for controlled protein delivery. The composite microspheres of a starch-based polymer and PLGA have been successfully formulated with spherical morphology, suitable particle size, high protein incorporation efficiency, and good protein stability. The system possesses sustained protein release and protein stabilization characteristics.

### EXAMPLE 2

### Microsphere System Containing Insulin

A novel composite microsphere delivery system comprised of poly(D,L-lactide-co-glycolide) (PLGA) and poly(acryloyl hydroxyethyl starch) (AcHES) hydrogel using bovine insulin as a model therapeutic protein was evaluated.

Insulin was incorporated into AcHES hydrogel microparticles by a swelling technique and then the insulin-containing AcHES microparticles were encapsulated in a PLGA matrix using a solvent extraction/evaporation method. The composite microspheres were characterized for loading efficiency, particle size and in vitro protein release. Protein stability was examined by SDS-PAGE, HPLC and MALDI-TOF MS. The hydrogel dispersion process was optimized to reduce the burst effect of microspheres and avoid hypoglycemic shock in the animal studies in which the serum glucose and insulin levels, as well as animal body weight, were monitored using a diabetic animal model.

Both the drug incorporation efficiency and the in vitro release profiles were found to depend upon preparation conditions. Sonication effectively dispersed the hydrogel particles in the PLGA polymer solution and the higher energy resulted in microspheres with a lower burst and sustained in vitro release. The average size of the microspheres was around 22 μm and the size distribution was not influenced by sonication level. HPLC, SDS-PAGE, along with MALDI-TOF MS, showed the retention of insulin stability in the microspheres. Subcutaneous administration of microspheres provided glucose suppression of <200 mg/dl for 8-10 days. During the treatment, the time points with higher serum insulin level were consistent with a more significant glucose suppression. Microsphere treated rats also grew virtually at the same rate as the normal control group until the insulin level declined and hyperglycemia returned. Multiple dosing given every 10 days demonstrated that the pharmacological effect and serum insulin levels from second or third doses were similar and comparable to that of the first dose.

Thus, the AcHES-PLGA composite microsphere system provides satisfactory in vitro and in vivo sustained release performance for a model protein, insulin, to achieve 10 day glucose suppression.

### EXAMPLE 3

### Preparation of Insulin Loaded Composite Microspheres and Evaluation of Same

Preparation of Insulin Loaded Composite Microspheres

Insulin loaded AcHES-PLGA composite microspheres were prepared. 50:50 PLGA Resomer RG502H was supplied by Boehringer Ingelheim (Ingelheim, Germany). Hydroxyethyl starch was obtained from Dupont Pharmaceutics (Wilmington, Del.). Acryloyl chloride was purchased from Aldrich Chemicals Company, Inc. (Milwaukee, Wis.). Bovine insulin (BI), polyvinyl alcohol (PVA, Mw 3000-7000), streptozotocin and Infinity™ glucose reagent were obtained from Sigma Chemical Co. (St. Louis, Mo.). The other reagents were of analytical grade. Insulin RIA kits were purchased from Linco Research, Inc (St. Charles, Mo.). Male Sprague Dawley rats were provided by Harlen (Indianapolis, Ind.).

Acryloyl hydroxyethyl starch (AcHES) was synthesized by esterifying hydroxyethyl starch (HES) with acryloyl chloride. AcHES hydrogel microparticles of around 0.5-2 μm were produced by free radical polymerization. The insulin loaded composite microspheres were prepared. To prepare a 1.5 g microsphere batch, 150 mg insulin in 0.75 ml 30% acetic acid was added to 101 mg of AcHES microparticles. The particles were allowed to swell for 5 minutes with vortex mixing. The polymer phase consisted of 1.25 g PLGA in 2.91 g methylene chloride (30% w/w). The polymer phase was added to the swollen AcHES particles, and either vortexed for 5 minutes or sonicated for 30 seconds at a predetermined power setting to form a dispersion (insulin in hydrogel)/(PLGA in methylene chloride). This primary dispersion was then added to 6% PVA solution and stirred by a Silverson mixer (Chesham, England) at 3000 rpm, and then transferred to 1 L deionized water for solvent extraction and evaporation. These procedures were conducted at about 4° Celsius using an ice bath. Then the temperature was gradually elevated to 39° Celsius to facilitate the removal of methylene chloride. Finally, the microspheres were washed with water and freeze-dried. Blank composite microspheres were fabricated in the same way without insulin.

The particle size of each batch was measured by laser scattering using a Malvern 2600 particle sizer (Malvem Instruments, England). The average particle size was expressed as the volume mean diameter in μm. The surface morphology and internal structure of fractured microspheres were examined by scanning electron microscopy (SEM) after palladium/gold coating.

Acetonitrile (ACN) was used to extract insulin from the microspheres (for the determination of drug loading efficiency and protein stability). Known amounts of insulin (1 mg or 2 mg) were mixed with blank AcHES/PLGA microspheres (18 or 19 mg) to mimic 5% and 10% loading. The mixture was dissolved in 4 ml of 90% acetonitrile (containing 0.1% TFA) with gentle shaking. Then 6 ml of 0.1% TFA in water was added to precipitate the polymer.

The supernatant was analyzed by HPLC using a phenomax-C18 column at room temperature with a binary gradient consisting of (A) 0.1% trifluoroacetic acid and (B) acetonitrile/water/TFA (90:9.9:0.1). The gradient consisted of 15% B to 75% B in 10 minutes, followed by equilibration at 15% B for 5 minutes. The peak elution was monitored at 220 nm.

Ten mg of each microsphere sample was analyzed using ACN extraction and HPLC to determine the loading efficiency.

Ten mg microspheres were hydrolyzed in 1 ml of 1 M NaOH with vigorous shaking at room temperature over night. Insulin standards were also hydrolyzed using the same procedure. After hydrolysis, 1 ml of 1.0 M HCl was added to neutralize the sample solutions. Insulin concentrations were determined by a micro-BCA total protein assay method.

The structural integrity of the bovine insulin extracted from the composite microspheres was characterized by sodium dodecyl sulfate poly(acrylamide) gel electrophoresis (SDS-PAGE). 5 mg of composite microspheres were boiled with the sample buffer containing 8% SDS and 0.2 M dithiothreitol (DTT) and then loaded onto a 16.5% tris-tricine SDS-PAGE gel after spinning down. The electrophoresis was performed at a constant voltage of 150 V. Protein bands on the gel were stained with GELCODE® Blue Stain Reagent.

10 mg microspheres were mixed with 0.4 ml 50:50 ACN:H₂O, vortexed and shaken for 30 minutes, centrifuged and the supernatant analyzed by MALDI-TOF MS. Spectra were obtained on a Kratos Kompact SEQ time-of-flight mass spectrometer (Manchester, UK), with ct-cyano-4-hydroxycinnamic acid as the matrix.

20 mg of microspheres were weighed and placed in 1.5 ml centrifuge tubes containing 10 mM glycine buffer (pH 2.8). The tubes were incubated at 37° C. At designated sampling times, the tubes were vortexed before centrifugation at 4000 rpm for 5 mm. The supernatant was collected and the volume of the release medium was restored with fresh glycine buffer. The samples were analyzed by HPLC.

Evaluation of the Efficacy of the Insulin Loaded Composite Microspheres In Vivo

The insulin composite microspheres were evaluated. Sprague Dawley male rats were injected intraperitoneally with 75 mg/kg of streptozotocin (40 mg/ml in 50 mM citrate buffer at pH 4.5). After 7 days the animals were anesthetized with ethyl ether after about 3-4 hours of fasting, and 0.7 ml of blood was collected from the tail vein. Rats with serum glucose levels higher than 500 mg/dl (analyzed by Infinity™ glucose reagent) were used for the experiments discussed below.

For single dose treatment, eight animals received the BI021 batch and six diabetic animals received the BI022 batch, at 345 IU insulin/kg (about 80 IU/rat) via subcutaneous injections at the neck region to simulate a dose of approximately 8 IU/day. The microspheres were suspended in an aqueous solution containing 1% carboxymethylcellulose sodium and 2% mannitol. The diabetic control group consisted of six animals without insulin treatment. At predetermined time points, 0.7 ml blood was collected after 3-4 hours fasting and the serum was assayed for glucose level (using Infinity™ glucose reagent) and insulin content by RIA (Linco Research, St. Charles, Mo.).

For multiple dosing treatment, eight diabetic rats were treated with 26 mg of microspheres (80 IU insulin) every 10 days. Blood was collected every other day until day 36. The control diabetic rats without insulin treatment were included.

Table 1 shows that the average particle size of the composite microspheres was 20.6-24.6 μm, suitable for parenteral administration through a 21 gauge needle. The particle sizes of each batch were similar and independent of the sonication power used for preparing the primary dispersion, as the final particle size depends mainly on the droplet size in the secondary emulsion and solidification rather than the size in the primary emulsion. Additionally, stirring speed, PLGA concentration in the organic phase and PVA concentration in the aqueous continuous phase were the same for each batch.

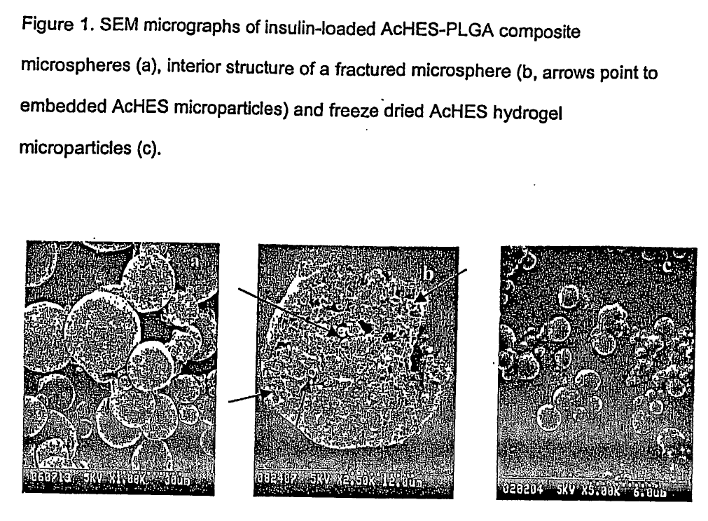

The microspheres prepared were spherical with relatively smooth surfaces (see FIG. 1a). FIG. 1b shows a fractured composite microsphere (from batch BI022), with the insulin loaded AcHES particles distributed throughout the matrix. The size of the AcHES particles was 0.5-2 μm (FIG. 1c), small enough for multiple particles to be encapsulated in a composite microsphere. The composite reduced the contact of protein molecules with the organic solvent during the emulsifying process which could contribute to protein denaturation and aggregation.

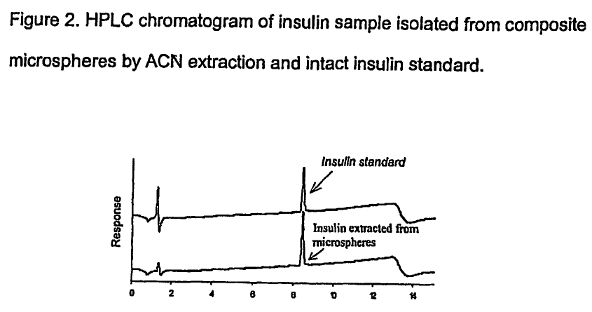

A typical RP-HPLC chromatogram is shown in FIG. 2, where the insulin sample from composite microspheres had an identical retention time as that of the insulin standard. This result provided supportive evidence that insulin did not degrade to products of different chemical nature. With the acetonitrile extraction, blank microspheres did not show any interference in the HPLC chromatograph and insulin spiked samples of blank microspheres had recoveries of 97.3-99.3% in all cases regardless of the type of PLGA or the amount of spiked insulin (5% or 10% w/w).

Characterization of Insulin Integrity in Composite Microspheres

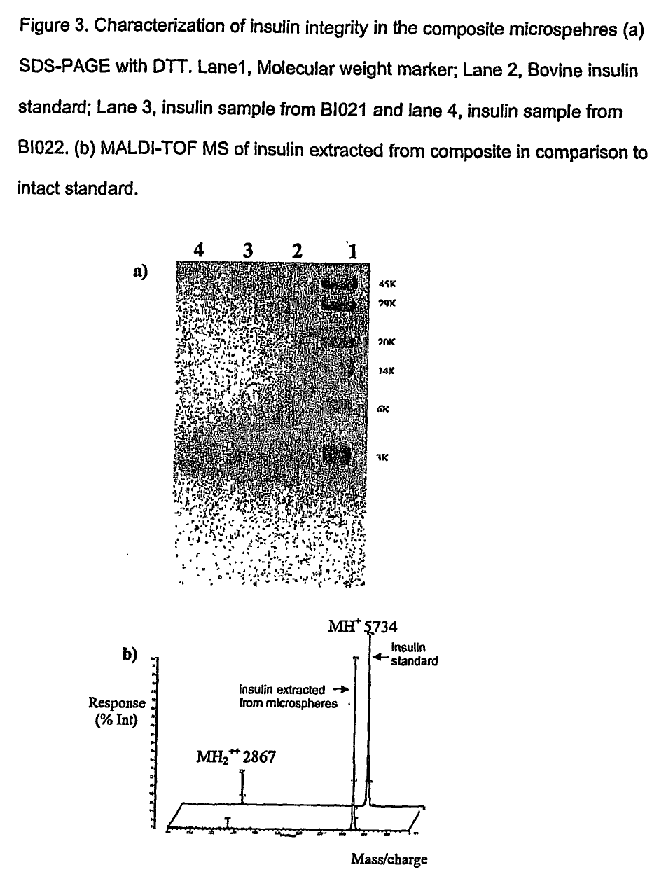

A SDS-PAGE (see FIG. 3a) of extracts from composite microspheres which were subjected to DTT reduction shows an insulin band at 3 Kda, corresponding to a mixture of A chain and B chain. No impurity band was found. In the MALDI-TOF mass spectrum (see FIG. 3b), insulin extracted from the microspheres displayed an identical monoisotropic peak at MH⁺ of 5734, with no evidence of covalent aggregation or degradation peaks. In contrast, forced degradation of insulin by DTT reduction showed no intact insulin peak and only separate peaks at MH⁺ of 3401 and 2338 for A chain and B chain, respectively. Therefore, insulin fragments could be detected using this extraction and MALDI-TOF MS as a stability indicating assay. Both the electrophoresis and mass spectrometry provided additional evidence for integrity of bovine insulin in the composite microspheres.

The Influence of Microsphere Preparation on Protein Incorporation and Release

Insulin incorporation efficiency was influenced by the preparation method of the primary emulsion. An efficiency of 76.5% was obtained by vortexing, whereas sonication yielded efficiencies >87.3% (see Table 1). The loading efficiency also increased slightly with a higher sonication power level, because vortex mixing at a lower power output resulted in large inner emulsion droplets which aided protein escape into the bulk aqueous phase when the secondary emulsion was prepared. At the higher sonication settings, to further disperse the hydrogel particles, a more uniform and finer primary emulsion was possible, resulting in a more effective incorporation of the protein.

Because insulin has a narrow therapeutic window, the burst release from the microspheres has to be low to avoid hypoglycemic shock and fatality in the animals. The sonication reduced the burst release of albumin by nearly one half. Table 1 shows that increasing the power level to disperse hydrogel in the polymer phase resulted in a significant decrease in the burst release. For example, batch BI018, prepared by vortexing, showed around 70% burst. Sonication reduced the burst to 5.07% and 1.86% at sonication output levels of 3.0 and 3.3, respectively. When the hydrogel was suspended in the polymer phase by vortexing or low energy level sonication (level 1.7), the resulting suspension did not appear very white and upon settling, hydrogel clumps were found on the wall of the vial. At sonication levels of 2.7, 3.0 and 3.3, the suspension appeared much whiter with no evidence of clumping, indicating a more uniform dispersion of the hydrogel particles.

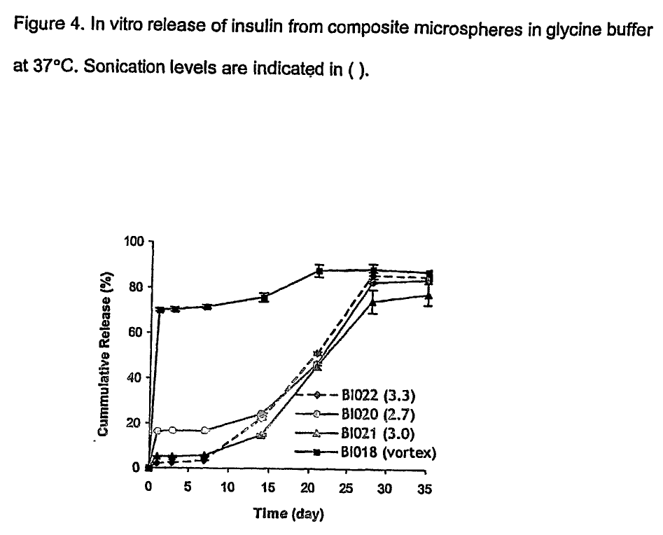

FIG. 4 shows the release profiles of insulin from four batches of composite microspheres prepared. The vortex batch had a 70% burst with slow subsequent insulin diffusion until erosion of polymer enhanced drug release after day 7. Due to the limited amount of insulin left within the microspheres, the release soon exhausted and reached a plateau. Three sonicated batches displayed similar release patterns except for the extent of initial burst. There was not much release from day 2 to 7 when protein diffuses through the incompletely hydrated matrix before polymer erosion occurs. The release accelerated during the second week due to polymer degradation and erosion and was even more rapid through the third and fourth weeks before the release plateaued after 30 days. Each batch released almost the entire incorporated drug (>78%).

In Vivo Evaluation of the Insulin Composite Microspheres

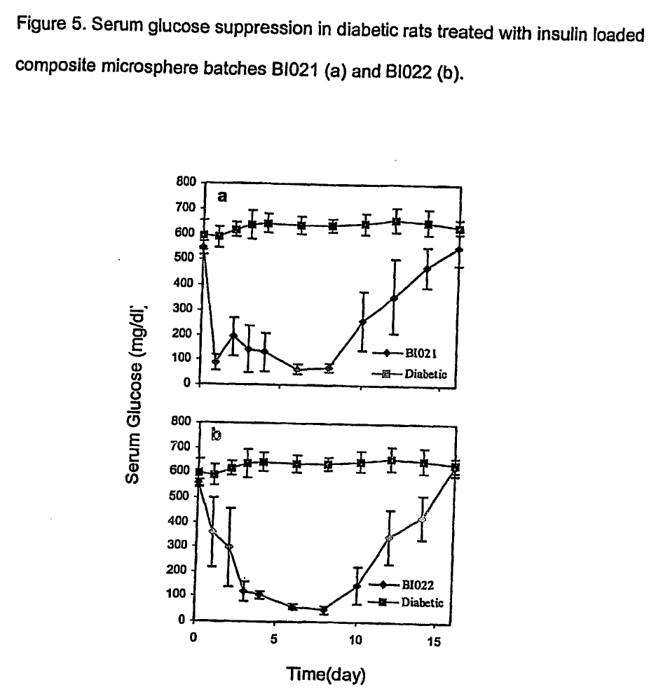

For single dose treatment, two composite microsphere batches, BI021 and Bl022, with 5.0% and 1.9% in vitro release on day 1, were selected for in vivo study. FIGS. 5a and 5b show the in vivo serum glucose profiles obtained upon subcutaneous administration of the two batches to diabetic rats. The rapid and immediate decrease in the serum glucose concentration observed on the very first day with batch BI021 seems in good agreement with it's higher initial in vitro release rate. On day 2, glucose level elevated to around 200 mg/dl but was still effectively suppressed compared to diabetic animals. The suppression was sustained through day 8 between 70 and 150 mg/dl with the most remarkable reduction seen on day 6. Hyperglycemia recurred after day 10 and returned to the diabetic control level on day 16. With respect to batch BI022, the suppression of glucose level was more gradual on the first 2 days most likely due to the lower burst. On day 3, glucose fell to around 130 mg/dl and the suppression was sustained for 10 days. After day 10, the level elevated to that of the diabetic control on day 16.

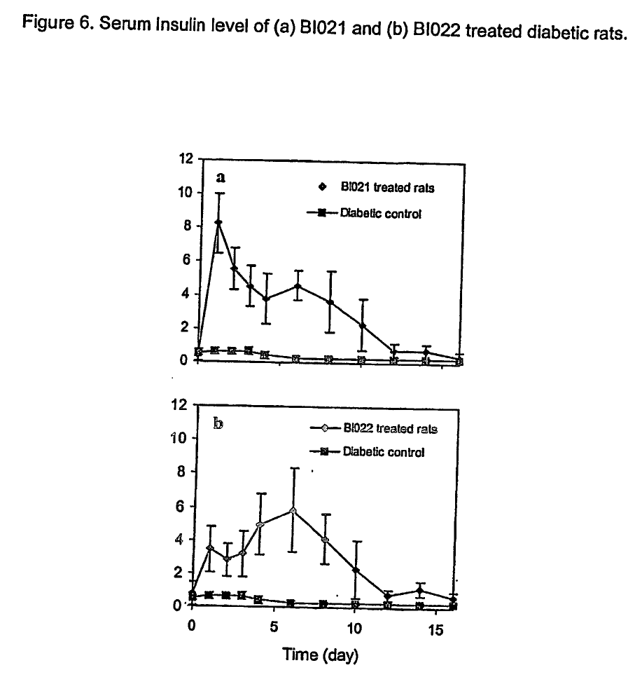

The serum insulin profiles seen in FIGS. 6a and 6b showed the insulin level in the two treated groups as well as the diabetic controls. In FIG. 6a, the administration of batch BI021 gave rise to an immediate peak on day 1, which correlated well with the rapid glucose suppression and the 5% in vitro burst release. Although the levels were in the 4-6 ng/ml range between 2 and 8 days, there is some evidence of a triphasic pattern, where the insulin level decreased on the second day and then elevated to a second peak on day 6. The decline of insulin level was gradual and there was steady insulin release from the composite microsphere up to day 10. In contrast, serum insulin levels of the control group showed a low level with no more than 0.7 ng/ml throughout the study. Treatment with BI022 led to a lower initial insulin level of 3.5 ng/ml within 24 hrs, which also correlated well with the 1.9% in vitro burst of this batch and the gradual suppression of blood glucose, seen in FIG. 5b. Three phases could be discerned with relatively lower insulin level on days 2 and 3, a peak of 5.8 ng/ml on day 6 and then a gradual decrease to original diabetic level after day 12.

Because a physiological response to insulin resulted in the form of growth of the animals and body weight increase, body weight of the BI021 and BI022 treated rats was monitored. The treated diabetic rats grew at a comparable rate to the control group of normal rats, until day 10 when hyperglycemia reappeared and loss of body weight reoccurred.

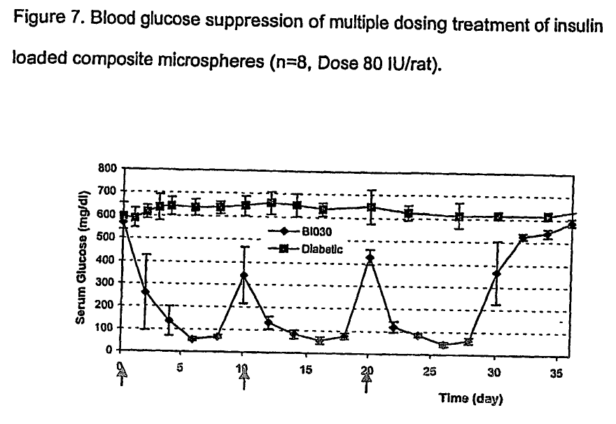

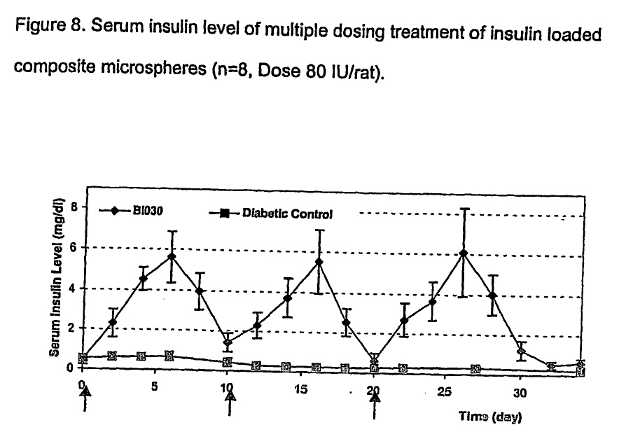

For multiple dose treatment, the object was to assess a multiple dose regimen for the model protein and evaluate the composite dosage form as a therapeutic application. As seen in FIG. 5b, the blood glucose elevated on day 10 after dosing. Therefore, rats (n=8) were treated with microspheres containing 80 IU insulin every 10 days. The profile of glucose shown in FIG. 7 from day 0 to day 10 was similar to that in FIG. 5b with maximum glucose suppression on day 6. The rate of glucose suppression was similar for the first and second doses, but because there were detectable residual levels of serum insulin upon administration of the second dose, the levels were suppressed to below 100 mg/dl faster (i.e., five days for the first dose, but only three days for the second dose). The second and third doses both exhibited prolonged and comparable pharmacological effect over 8 days. The result suggests that 10 days may be an appropriate dosing schedule because once the blood glucose elevates beyond normal level, a subsequent dose could take over and keep the glucose level in control. The in vivo insulin level (see FIG. 8) also displayed comparable pharmacokinetic profiles from the three doses with regard to Cₘₐₓ Tₘₐₓ and AUC.

Thus, bovine insulin was successfully encapsulated into the composite microspheres with retention of insulin integrity, and the microsphere preparation process was optimized to reduce the burst and provide in vitro sustained release. An extraction and HPLC analytical method was developed to simultaneously determine insulin loading and protein stability. The glucose suppression in diabetic rats was prolonged through 8-10 days with the most remarkable reduction seen on days 6-8. Multiple dosing reflected the repetitive pharmacological efficacy and pharmacokinetic profile of a single dose. The in vitro and in vivo results show that the novel composite microsphere system could be used as a vehicle for prolonged delivery of protein drugs.

TABLE 1
Batch summary of insulin loaded composite microspheres.

| | Batch | Sonication | | Loading | Burst | Average |
|---|---|---|---|---|---|---|
| | size | output | Loading | efficiency | Release | Size |
| Batch | (g) | Level* | % | % | (%) | (μm) |
| BI018 | 2.0 | Vortex | 7.65 | 76.5 | 69.2 | 23.8 |
| BI019 | 2.0 | 1.7 | 8.73 | 87.3 | 60.1 | 20.6 |
| BI020 | 1.5 | 2.7 | 9.59 | 95.9 | 16.2 | 24.6 |
| BI021 | 1.5 | 3.0 | 9.94 | 99.4 | 5.07 | 22.1 |
| BI022 | 1.5 | 3.3 | 10.1 | 101.1 | 1.86 | 21.3 |

*Vortexing and sonication time was 5 minutes and 30 seconds, respectively.

### EXAMPLE 4

### Preparation and Characterization of Poly(D,L-lactide-co-glycolide) Microspheres for Controlled Release of Human Growth Hormone

Human growth hormone (HGH), a single polypeptide chain of 191 amino acid residues with a molecular mass of 22 kDA, is a somatotropic hormone secreted from the anterior pituitary gland. HGH may be used to treat chronic renal insufficiency, Turner's syndrome and cachexia secondary to AIDS.

When producing microencapsulated formulations of therapeutic proteins, it is important that the physical, chemical, and biological properties of the protein remain intact during encapsulation. It is particularly important to preserve protein structure and bioactivity and not impart any immunogenicity. In fact, antibody responses can lead to safety concerns and, if neutralizing, can limit the efficacy of subsequent treatment. To overcome these problems, one approach is the physical encapsulation of protein loaded hydrophilic particles or hydrogels into a PLGA matrix.

In this study, hydrophilic starch-based hydrogel particles containing rHGH were prepared by a simple swelling procedure. The rHGH-loaded hydrogel particles were then encapsulated in PLGA microspheres to form the hydrogel-PLGA combined composite microspheres, using a solvent extraction/evaporation method. rHGH loaded PLGA microspheres were prepared as well by the same solvent extraction/evaporation method, using mannitol instead of starch hydrogel microspheres. T aim of this study was to assess the physicochemical characteristics and in vitro protein release of both microsphere formulations and to establish poly (acryloyl hydroxyethyl starch)-PLGA (acHES-PLGA) composite microspheres as a protein delivery system in comparison with the mannitol containing PLGA microspheres. The physical integrity of rHGH in both microsphere batches was assessed.

PLGA with free carboxyl end groups was purchased from Boehringer Ingelheim (Boehringer Ingelheim, Germany, RG502H). Hydroxyethyl starch [Hetastarch (HES)] was obtained from Dupont Pharmaceuticals (Wilmington, Del.) and Acroyl chloride was purchased from Aldrich Chemicals Company, Inc. (Milwaukee, Wis.). rHGH was obtained from Dong-A Pharm. Co., Ltd. (Kyunggi, Korea). Polyvinyl alcohol (PVA) was obtained from Sigma Chemical Co. (St. Louis, Mo.), a micro-BCA total protein assay kit was from Pierce (Rockford, Ill.), and mannitol was from Fisher (Nepean, Ontario).

Preparation of PLGA-AcHES Composite Microspheres

Acrylic acid ester of hydroxyethyl starch (acHES) was prepared. The PLGA-acHES composite microspheres were prepared by a modified solvent extraction/evaporation method with 10% target loading of rHGH. 58.1 mg rHGH powder was dissolved in 0.40 mL of 0.1 M PBS (pH 7.4). The protein solution was added to acHES particles (10% of total polymer weight), and the particles were allowed to swell for 5 minutes with vortex mixing at room temperature.

A 30% (w/w) PLGA methylene chloride solution was added to the swollen acHES particles and vortexed for 3 minutes at room temperature to form a (protein in hydrogel)/(polymer in solvent) dispersion. This primary dispersion was then added to precooled (4° C.) 100 mL 6% PVA solution and stirred by a Silverson mixer (Silverson, Chesham Bucks, England) at 2500 rpm for 1 minute. The resulting secondary suspension was transferred to 1 L deionized water and stirred gently for 3 hours at room temperature to remove the organic solvent and solidify the polymer. The microspheres were filtered and freeze-dried.

Preparation of Mannitol PLGA Microspheres

Mannitol PLGA microspheres were prepared by the modified solvent extraction/evaporation method with 10% target loading of rHGH. A primary dispersion was prepared by mixing the protein solution containing 10% mannitol with 30% PLGA solution and then the emulsion was added to 6% PVA solution while stirring at 200 rpm. The resultant suspension was transferred to 1 L deionized water and stirred gently for 3 hours at room temperature to remove the organic solvent and solidify the polymer. The microspheres were filtered and freeze-dried and stored at 4° C.

Microsphere Characterization

The morphology and size of the microspheres were analyzed by scanning electron microscopy (SEM) (Hitachi Model S800) and laser light diffraction (Malvern Instruments, UK). To determine drug content, triplicate samples of 5 mg of microspheres were dissolved in 0.5 mL 1 M NaOH by overnight rotation. Then the solution was neutralized with 0.5 mL 1 M HCl. rHGH content in the samples was determined by Micro-BCA protein assay.

In vitro release

The in vitro hGH release was determined by suspending 15 mg microspheres in 1 ml of PBS (pH: 7.4) and gently rotating at 37° C. At regular intervals, samples were centrifuged and the supernatant was removed for Micro-BCA protein assay. Fresh replacement media was added to resuspend the microspheres. The analysis was performed in triplicate.

Dry and Rehydrated Protein FTIR Analysis

The secondary structure of rHGH was investigated either in the rHGH native protein and freeze-dried powder or after entrapment in PLGA with mannitol and acHES-PLGA composite microspheres. Infrared spectra were obtained by using a Bio-Rad Excalibur FTS 3000 MX spectrometer equipped with a DTGS KBr detector, with a 0.25 cm⁻¹ maximum resolution and signal-to-noise ratio 25,000:1. Analyses on dried polymer and microspheres were performed on samples prepared by mixing an amount equal or correspondent to 1 mg of rHGH to 200 mg of KBr and annealed into disks. This process does not modify the spectrum profile of dry proteins. A background spectrum consisting of blank KBr was previously collected. For all spectra 256 consecutive scans were collected in a single beam mode with a 2 cm⁻¹ resolution. A reference spectrum of PLGA polymer was recorded under identical conditions and the protein spectra were obtained by subtraction of the reference spectrum. Spectra of native protein solution, dissolved freeze-dried powder and rehydrated microspheres were recorded in a CaF₂ windows cell with a 6 μm spacer. Microspheres were incubated in PBS 0.1 M for 4 hours at 37° C. and immediately analysed. Background and reference spectra consisting in air, buffer and rehydrated blank microspheres respectively were recorded as well and properly subtracted from the sample spectra to eliminate vapor, buffer and polymer contributions.

The obtained curves were employed in order to compare the amide I region profiles of the samples with the standard rHGH profile. The evaluation of the protein secondary structure retention was accomplished by applying an 11-points Savitzky-Golay smoothing function to eliminate noise and operating the second derivative transformation on the subtracted spectra. These curves were imported in a SYSTAT's Peakfit® v. 4.11 software and fitting was performed, applying a two points baseline, in order to assess the percent of retention of the dominant α-helix band in the amide I region. rHGH secondary structure retention was expressed as percent of the normalized area of the peak at ca. 1655 cm⁻¹ with respect to protein standard. The standard consisted in the native rHGH solution.

Characterization of Microspheres

To stabilize the protein during microsphere encapsulation process and within the microspheres after hydration, the protein was formulated with the acHES particles. The acHES particles for protein entrapment possess a submicrometer mean particle size of 0.14 μm, a low bulk density of 0.05 g/cc, and high specific surface area In addition, the acHES hydrogel particles showed fast and good swelling properties. The hydrated particles showed about an 11-fold larger particle diameter and were 10.5-fold heavier than the dry particles. These results suggest that the hydrophilic starch-based hydrogel could imbibe inside a large amount of aqueous drug solution and protect the drug from degradation due to solvent and polymer interactions during microsphere preparation and drug release.

The encapsulation in PLGA microspheres was performed with aqueous protein solution containing mannitol as an alternate means of stabilizing rHGH during microparticle preparation. Previous studies with sugars and proteins demonstrated that sugars cause preferential hydration of proteins, resulting in stabilization of the compact native state. The formation of a hydration layer around the compact protein may reduce the protein-organic solvent interactions preventing protein denaturation.

As shown in Table 2, the average particle size of the composite microsphere was 44.6±2.47 μm. The conventional PLGA-mannitol microspheres showed an average particle size of 39.7±2.50 μm. rHGH was encapsulated successfully in the composite and PLGA-mannitol microspheres with 93.2% to 104% drug loading efficiency. The protein incorporation efficiency increased with increasing PLGA polymer concentration in the disperse phase and PVA in the continuous phase. Higher viscosity achieved by increasing polymer and PVA concentration could minimize diffusion of protein from the disperse phase to continuous phase during the fabrication of microspheres and may also have resulted in more condensed PLGA matrices around entrapped aqueous protein droplets.

In vitro release

PLGA-acHES composite loaded microspheres exhibited a high burst effect with 50% hGH release after one day compared to the PLGA with mannitol microspheres. In general, the release of rHGH from PLGA-acHES composite and PLGA with mannitol microspheres occurs by two mechanisms. The strong burst effect observed for PLGA-acHES composite microspheres is due to diffusion of rHGH from acHES hydrogel particles located near the microsphere surface, through the channels and inner pores formed by solvent removal during the microspheres solidification process. The second mechanism involves the degradation and solubilization of the PLGA matrix. Compared to the composite microspheres, the conventional PLGA with mannitol microspheres showed about 15% less initial release followed by a progressive release for 41 days. As shown by mass balance investigation at the end of 41 days, rHGH was completely released from PLGA-acHES composite microspheres. Compared to the PLGA with mannitol microspheres, dissolution of PLGA domains in the composite microspheres could expose the entrapped rHGH-containing acHES hydrogel particles to the release media, and the exposed hydrogel could release more rHGH molecules with little or no interaction with the PLGA polymer. As a result, the composite microspheres showed more favorable in vitro release than the conventional PLGA with mannitol microspheres for rHGH drug delivery.

Secondary Structure Evaluation by FTIR

The use of FTIR technique for the detection of secondary structure changes in proteins, in particular for rHGH is known. The bands at around 1656 cm⁻¹ and 1631 and 1695 cm⁻¹ in the amide I region (1600-1700 cm⁻¹) have been identified as carbonyl stretching of α-helices and β-sheets respectively, which characterize the secondary structure of the protein. Several other peaks contribute to the broad amide I band and these modes have been associated with the vibration frequencies of the disordered internal structure elements, such random coils, extended chains and β-turns. In particular, the relative high α-helix content of rHGH is important in the study of the encapsulation procedure effect on the structure of the protein.

Analyses were performed either on dry and rehydrated samples. This procedure is required since the protein profile in the dry state may not be predictive of that obtained after rehydration. In fact, according to the chemical-physical properties of the molecule, many factors, such as excipients, pH and aggregation may cause non-reversible unfolding of the protein. Therefore, scans of dry and rehydrated samples were performed under identical conditions. Spectra of dry rHGH protein and microspheres were obtained, after subtraction of the PLGA reference spectrum. Similar treatment was employed for rehydrated microspheres analysis. In this case, rehydrated blank microspheres spectrum was employed as a reference. α-helix band retention evaluation was accomplished by comparison of relative percent areas of the peaks of the rehydrated samples at ca. 1655 cm⁻¹ that can be deduced by curve-fitting of the second derivative spectra in the range 1600-1700 cm⁻¹.

Table 3 lists the calculated normalized % areas of dry samples obtained for α-helix and β-sheet components. The comparison of the obtained profiles with the profile of the native protein shows the broadening effect as a result of the induced structural changes and solid-state aggregation. This effect is less dramatic for rHGH entrapped in microspheres. Additionally, the calculated amide I α-helix content was comparable for all samples and a higher percent value (33%) was found for the composite microspheres, whereas the β-sheet contribution increased for the microspheres (21% for the composite) compared to the freeze-dried powder. The main contribution was found for unordered structures like coils, turns and extended chains.

On the other hand, the profile obtained upon rehydration of the dry samples showed an increase in the α-helix component due to partial refolding of the protein in solution. α-helix % areas were 47% for composite microspheres, 24% for microspheres with mannitol and 38% for the lyophilized protein. The corresponding retention of the α-helix band at 1655 cm⁻¹ was close to 80% for the composite batch, only 40% for PLGA microspheres with mannitol and 63% for freeze-dried rHGH as compared to the standard band area. In addition, although β-sheets content remained almost the same, the high presence of coils, turns and extended chains was now reduced and the lowest contribution was found for the composite microspheres with only 33% of the total. The low α-helix value for PLGA microspheres with mannitol may be correlated with the presence of non-native aggregates of the protein as shown by the 26% β-sheets contribution to the total amide I band that it is usually found to be representative of protein aggregation.

These findings infer that rHGH entrapment in acHES-PLGA composite microspheres caused much less modifications on the protein secondary structure during the preparation process than for PLGA-mannitol microspheres and freeze-dried powder. The starch environment seems to exert an effective protective action on the entrapped rHGH perhaps by avoiding direct contact with the surrounding dichloromethane phase during the microsphere formation process.

Thus, a biodegradable microsphere system has been developed for controlled rHGH delivery. The composite microspheres of a starch-based polymer and PLGA have been successfully formulated with spherical morphology, high protein incorporation efficiency and good stability. The system possesses sustained rHGH release and rHGH protein stabilization characteristics. The protein was effectively protected by the starch environment when entrapped in acHES-PLGA composite microspheres. The structure of the protein in dried microspheres may be predictive of storage stability of the protein. AcHES-PLGA microspheres are biodegradable and may be useful for the in vivo delivery of rHGH.

TABLE 2
Characterization of rHGH loaded microspheres.

| | | Loaded | Encap- | |
|---|---|---|---|---|
| | Target | microspheres | sulation | Average |
| | loading | hGH content | efficiency | particle size |
| Microspheres | (%) | (%) ± S.D. | (%) ± S.D. | (_m) ± S.D. |
| PLGA- | 10 | 10.39 ± 0.09 | 103.9 ± 0.88 | 44.6 ± 2.47 |
| acHES | | | | |
| PLGA with | 10 | 9.32 ± 0.09 | 93.2 ± 0.94 | 39.7 ± 2.50 |
| mannitol | | | | |

TABLE 3
Calculated data for α-helix and β-sheets
components in the amide I region for standard,
freeze-dried and encapsulated rHGH dry samples.

| Secondary | _-helix | _-sheet | Other^{a} |
|---|---|---|---|
| structure | % Normalized | % Normalized | % Normalized |
| content | Area ± S.D. | Area ± S.D. | Area ± S.D. |
| Standard rhGH | 61 | 14 | 25 |
| in solution | | | |
| rhGH dry | 29 ± 2 | 10 ± 1 | 61 ± 2 |
| Dry MS with | 21 ± 1 | 14 ± 1 | 59 ± 2 |
| mannitol | | | |
| Dry composite | 33 ± 1 | 21 ± 1 | 46 ± 1 |
| MS | | | |

^{a}Includes random coils, turns, extended chains

TABLE 4
Percent of α-helix structure and β-sheet after rehydration
of rHGH free and entrapped in microspheres with mannitol and composite
microspheres. α-helix retention was calculated respect to the
normalized peak area. Increase in percent of the β-sheets component
is evident respect to the standard.

| | _-helix | _-sheet | Other^{a} | |
|---|---|---|---|---|
| | % | % | % | |
| Secondary | Normalized | Normalized | Normalized | % _-helix |
| structure | Area ± | Area ± | Area ± | retention ± |
| content | S.D. | S.D. | S.D. | S.D. |
| Standard | 61 | 14 | 25 | — |
| Rehydrated | 38 ± 1 | 10 ± 1 | 52 ± 2 | 63 ± 2 |
| freeze-dried | | | | |
| powder | | | | |
| Rehydrated | 24 ± 1 | 26 ± 2 | 50 ± 2 | 40 ± 2 |
| MS with | | | | |
| mannitol | | | | |
| Rehydrated | 47 ± 1 | 20 ± 1 | 33 ± 1 | 77 ± 2 |
| composite | | | | |
| MS | | | | |

^{a}Includes random coils, turns, extended chains

### EXAMPLE 5

### A Non-Invasive Protein Extraction Technique for Protein Encapsulated Poly(lactide-co-glycolide) (PLGA) Microspheres

A non-invasive protein extraction technique for protein encapsulated poly(lactide-co-glycolide) (PLGA) microspheres was developed and validated for drug content determination and protein stability assessment. Although accurate protein content may be achieved utilizing radiolabelled protein, two alternative extraction techniques have been reported. One involves the dissolution of the microspheres in methylene chloride (MC) followed by aqueous extraction of the protein and the other is alkaline hydrolysis of the MS, followed by protein assay. These techniques may provide dependable quantitation of protein loading but fail to provide information on stability because of protein denaturation induced by the extraction process; the former due to the organic solvent and W/O interface and the latter due to protein digestion in the alkaline solution. This study presents a strategy for protein extraction to enable both protein content determination and stability assessment. Lysozyme was used as a model protein.

Recovery: 1 mg lysozyme was added to 19 mg bland PLGA microspheres to mimic 5% loading. Three ml polyethylene glycol (PEG) 400 containing 5% glycine buffer (0.1 M, pH 2.0) was used to dissolve the MS. The polymer was then precipitated with the addition of ˜7 ml glycine buffer and the supernatant removed for HPLC assay.

PEG method: Lysozyme loaded MS were extracted and the supernatant was used for enzymatic activity analysis, SDS-PAGE, circular dichroism (CD) and DSC.

MS prepared from 5 different polymers showed good recoveries (99.4-100.5%) of spikes lysozyme (Table 5).

TABLE 5
Recovery of blank MS spiked with lysozyme

| Lysozyme | PLGA type | Blank MS | Recovery (%) |
|---|---|---|---|
| 1 mg | 501H | 19 mg | 100.2 ± 0.4 |
| 1 mg | 502H | 19 mg | 99.4 ± 0.9 |
| 1 mg | 503H | 19 mg | 100.5 ± 1.3 |
| 1 mg | 502 | 19 mg | 100.4 ± 0.6 |
| 1 mg | 503 | 19 mg | 99.8 ± 1.5 |

TABLE 6
Extraction of protein loaded microparticles.

% Loading Measured

| MS | MC 1 ext. | MC 2 ext. | NaOH | PEG |
|---|---|---|---|---|
| 501H | 4.00 | 5.50 | 7.82 | 8.02 |
| 502H | 5.62 | 7.73 | 10.20 | 10.36 |
| 503H | ND | ND | 5.49 | 4.17 |
| 502 | 2.70 | 4.14 | 5.11 | 5.98 |
| 503 | ND | ND | 1.72 | 1.82 |

As seen in Table 6, MC extraction gave much lower protein content than the other 2 methods. The two-phase extraction (MC and buffer) was not complete and gave rise to an underestimation of drug content. Although there were slight differences in the PEG and NaOH extractions for some batches, these methods were found to generate comparable results.

The extracted protein sample using the PEG method was applied to various stability assessments, including enzymatic activity analysis, SDS-PAGE, CD and DSC. The recovered lysozyme from the microsphere batches had good retention of enzymatic activity. (Table 7).

TABLE 7
Enzymatic activity of encapsulated lysozyme.

| Batch | Specific Activity Retained (%) |
|---|---|
| 501H | 93.2 ± 4.9 |
| 502H | 97.7 ± 0.3 |
| 503H | 101.5 ± 2.6 |
| 502 | 91.8 ± 3.2 |
| 503 | 92.7 ± 1.2 |

While this invention has been particularly shown and described with references to preferred embodiments thereof, it will be understood by those skilled in the art that various changes in form and details may be made therein without departing from the spirit and scope of the invention as defined by the appended claims.

All references discussed above are herein incorporated by reference in their entirety.

We claim:

1. A composite microsphere system comprising
poly(D,L-lactide-co-glycolide) (PLGA);
poly(acryloyl hydroxyethyl starch) (AcHES); and
a pharmaceutically effective amount of a biologically active compound;
wherein the biologically active compound is a polypeptide having a molecular weight of about 200 to about 160,000 Daltons.

2. The composite microsphere system of claim 1, wherein the biologically active compound is selected from the group consisting of an insulin, an interferon, a luteinizing hormone-releasing hormone (LHRH) analog, a somatostatin and/or somatostatin derivative, a calicitonin, a parathyroid hormone (PTH), a bone morphogenic protein (IMP), an erythropoietin (EPO), an epidermal growth factor (EGF) and a growth hormone.

3. A drug formulation comprising a composite microsphere system comprising
poly(D,L-lactide-co-glycolide) (PLGA);
poly(acryloyl hydroxyethyl starch) (AcHBS); and
a pharmaceutically effective amount of a biologically active compound;
wherein the biologically active compound is selected from the group consisting of an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone; and
a pharmaceutically acceptable vehicle.

4. A method for the sustained release delivery of a therapeutic compound to a subject comprising:
administering to the subject a composite microsphere system comprising
poly(D,L-lactide-co-glycolide) (PLGA);
poly(acryloyl hydroxyethyl starch) (AcHES); and
a pharmaceutically effective amount of a biologically active compound;
wherein the biologically active compound is selected from the group consisting of an insulin, an interferon, a luteinizing hormone-releasing hormone (LHRH) analog, a somatostatin and/or somatostatin derivative, a calicitonin, a parathyroid hormone (PT), a bone morphogenic protein (BMT), an erythropoietin (EPO), an epidermal growth factor (EGF) or a growth hormone.

5. The method of claim 4, wherein the subject is suffering from a condition which can be treated by the administration of a biologically active compound selected from the group consisting of an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone.

6. The method of claim 4, wherein the subject is a vertebrate or an invertebrate organism.

7. The method of claim 4, wherein the subject is a canine, a feline, an ovine, a primate, an equine, a porcine, a caprine, a camelid, an avian, a bovine, an amphibian, a fish, or a murine organism.

8. The method of claim 4, wherein the primate is a human.

9. The method according to claim 4, wherein the drug is administered intramuscularly.

10. The method of claim 4, wherein the microspheres are in a pharmaceutically acceptable vehicle.

11. The method of claim 4, wherein the microspheres are administered topically.

12. The method of claim 11, wherein the topical administration is via inhalation or nasal administration.

13. The method of claim 4, wherein the microspheres are administered parenterally.

14. A method of preparing a composite microsphere system of claim 2, comprising
incorporating a biologically active ingredient selected from the group consisting of an insulin, an interferon, a luteinizing hormone-releasing hormone (LHRH) analog, a somatostatin and/or somatostatin derivative, a calicitonin, a parathyroid hormone (PTH), a bone morphogenic protein (BMP), an erythropoietin (EPO), an epidermal growth factor (EGF) or a growth hormone into AcHES hydrogel microparticles; and
encapsulating the resulting AcHES hydrogel microparticles containing the biologically active ingredient into a PLGA matrix.

15. The method of claim 14, wherein the AcHES hydrogel microparticles containing the biologically active ingredient are incorporated into the PLGA matrix using a process selected from the group consisting of solvent extraction, solvent evaporation, spray drying, freeze drying and a combination thereof.
